(19) United States
(12) Patent Application Publication
(10) Pub. No.: US 2022/0309198 A1
(43) Pub. Date: Sep. 29, 2022

(54) Sample Preparation Evaluation System, Sample Preparation Evaluation Method and Computer Readable Medium

(71) Applicant: TOKYO INSTITUTE OF TECHNOLOGY, Tokyo (JP)

(72) Inventors: Taro HITOSUGI, Tokyo (JP); Ryota SHIMIZU, Tokyo (JP)

(21) Appl. No.: 17/638,544

(22) PCT Filed: Aug. 26, 2020
(86) PCT No.: PCT/JP2020/032101

§ 371 Date: Feb. 25, 2022

(30) Foreign Application Priority Data
Sep. 5, 2019 (JP) 2019-162209
Jul. 31, 2020 (JP) 2020-130737

(51) Int. Cl. G06F 30/10

(52) U.S. Cl. CPC G06F 30/10

(57) ABSTRACT

A sample preparation evaluation system comprises a preparation device that prepares a sample, a measurement device that measures a physical property or a structure of the sample prepared by the preparation device and outputs substance information indicating the measured physical property or the measured structure of the sample and a prediction device connected to the preparation device and the measurement device. The prediction device predicts a preparation condition for optimizing the substance information based on a data set including the preparation condition of the sample and the substance information of the sample. The preparation device prepares a sample according to the predicted preparation condition. The prediction device adds the substance information of the sample measured by the measurement device and the preparation condition of the sample to the data set, and sequentially predicts the preparation condition based on the data including the added preparation condition and the added substance information.

### CROSS-REFERENCE TO RELATED APPLICATIONS

This application is the national phase under 35 U. S. C. § 371 of PCT International Application No. PCT/JP2020/032101 which has an International filing date of Aug. 26, 2020 and designated the United States of America.

### FIELD

The present invention relates to a sample preparation evaluation system, a sample preparation evaluation method and a computer readable medium.

### BACKGROUND

There is an effort called materials informatics to efficiently search for a material in combination with an information processing technique such as data mining or the like when research or development of a new material or an alternative material is performed.

Following this, various simulation methods have been proposed of searching for design and development of an optimum material in a data space.

### SUMMARY

The current state of the materials informatics merely predicts an optimum material and leaves prompt preparation of the predicted material and development to a practical material to the human work.

In one aspect, the object is to provide a sample preparation evaluation system that is capable of suitably implementing materials informatics.

A sample preparation evaluation system according to one aspect comprises a preparation device that prepares a sample, a measurement device that measures a physical property or a structure of the sample prepared by the preparation device and outputs substance information indicating the physical property or the structure of the sample prepared by the preparation device and a prediction device connected to the preparation device and the measurement device. The prediction device predicts a preparation condition for optimizing the substance information based on a data set including the preparation condition of the sample and the substance information of the sample, the preparation device prepares the sample according to the preparation condition predicted by the prediction device, the prediction device adds the substance information of the sample and the preparation condition of the sample to the data set, and the prediction device sequentially predicts the preparation condition based on the data set including the added preparation condition and the added substance information.

### BRIEF DESCRIPTION OF THE DRAWINGS

FIG. 1 is a schematic view showing an example of the configuration of a preparation evaluation system.

FIG. 2 is a block diagram showing an example of the configuration of a prediction device.

FIG. 3 is a block diagram showing an example of the configuration of a server.

FIG. 4 is an explanatory view showing an example of the record layout of a sample DB.

FIG. 5 is an explanatory view showing an outline of Embodiment 1.

FIG. 6 is a flowchart showing an example of the processing procedure executed by the prediction device.

FIG. 7 is schematic view showing an example of the configuration of a preparation evaluation unit according to Embodiment 2.

FIG. 8 is a schematic view showing an example of the configuration of a preparation evaluation system according to Embodiment 3.

FIG. 9 is an explanatory view showing an outline of Embodiment 3.

FIG. 10 is an explanatory view showing an example of a display screen of sample information.

FIG. 11 is a flowchart showing an example of the processing procedure to be executed by the server.

FIG. 12 is experimental data showing the oxygen partial pressure dependence of electric resistance.

### DETAILED DESCRIPTION

Hereinafter, the present invention will be described in detail based on the drawings showing embodiments thereof.

### Embodiment 1

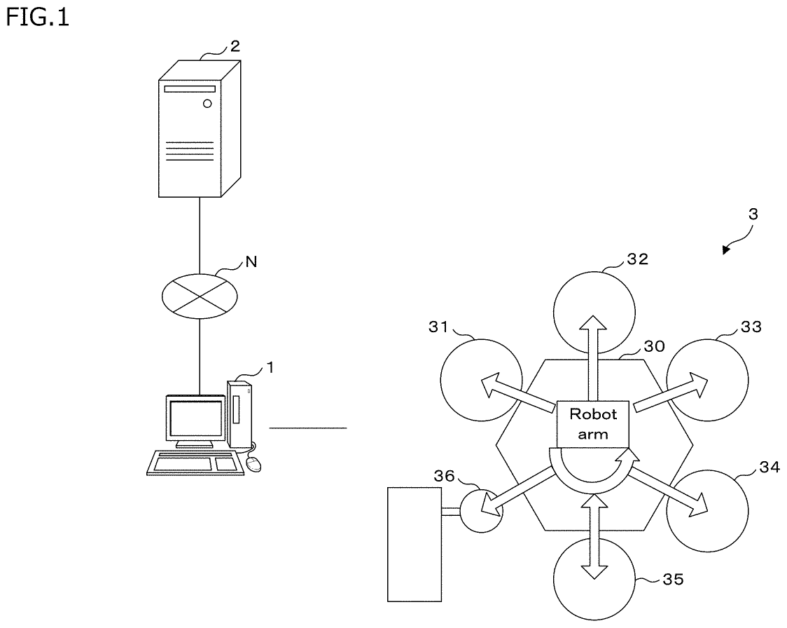

FIG. 1 is a schematic view showing an example of the configuration of a preparation evaluation system. In the present embodiment, a preparation evaluation system will be described that automatically prepares, measures and optimizes a sample for research and development of a new material and an alternative material. The preparation evaluation system includes a prediction device 1, a management device 2 and a preparation evaluation unit 3. The prediction device 1 and the management device 2 are connected so as to communicate with one another through a network N.

The prediction device 1 is an information processing apparatus capable of performing various kinds of information processing and transmission and reception of information, and is, for example, a personal computer or a server computer. In the present embodiment, the following description is made assuming that the prediction device 1 is a personal computer. The prediction device 1 performs processing of predicting a preparation condition of a sample having optimum substance information (physical property or structure) from a dataset including preparation conditions of multiple samples and substance information indicating the physical properties or the structures of the samples. In the present embodiment, the prediction device 1 performs sequential searches for a preparation condition that maximizes (or minimizes) a predetermined physical property value (substance information) using a method of Bayesian optimization as described below.

It is noted that “prediction” is herein synonymous with “search” for the convenience of explanation. Furthermore, “data set” indicates a group of data consisting of pieces of data on multiple samples used in the same experiment (Bayesian optimization processing). Moreover, data on each of the samples is called “sample information” so as to be discriminated from “data set.”

In the present embodiment, the prediction device 1 not only predicts the preparation condition of an optimum sample but also inputs the predicted preparation condition to the preparation evaluation unit 3 to cause the preparation evaluation unit 3 to prepare a sample according to the preparation condition. The prediction device 1 causes the preparation evaluation unit 3 to measure a physical property value of the prepared sample and adds to the data set the measured physical property value and the preparation condition of the sample as a target to be measured. The prediction device 1 performs sequential searches for the preparation condition of an optimum sample every time the data set is added and inputs the preparation condition of the optimum sample to the preparation evaluation unit 3 to cause the preparation evaluation unit 3 to prepare a sample. The prediction device 1 repeats this processing to search for an optimum sample.

As one example of the present embodiment, the case where a thin-film material for a semiconductor is deposited will be described. More specifically, the case where the ionic conductivity (electrical conductivity) of the thin-film material is optimized will be described assuming that a thin-film material of Li ion solid electrolyte is deposited.

The management device 2 is a management device that manages information on each of the samples prepared by the preparation evaluation unit 3 and is a server computer functioning as a database server. In the following description, the management device 2 is read as a server 2 for convenience's sake. The server 2 acquires information on samples (data set) prepared in the preparation evaluation unit 3 from the prediction device 1 and stores (saves) and manages it in a database.

In the present embodiment, though the following description will be made assuming that the prediction device 1 that searches for an optimum sample and the server 2 that manages sample information are regarded as separate computers, the prediction device 1 and the server 2 may be configured as a single computer. Alternatively, the prediction device 1 may be an external computer connected so as to communicate with the preparation evaluation unit 3 via a network N as in the server 2.

The preparation evaluation unit 3 is a device for preparing a sample according to a preparation condition input from the prediction device 1 and is a deposition device for depositing a thin-film material as described above. In the present embodiment, the preparation evaluation unit 3 is a cluster-type sputtering device and is disposed with multiple chambers in a polygonal (hexagonal) shape. The preparation evaluation unit 3 includes a transfer device 30, deposition chambers 31, 32, 33 (preparation device), a measurement chamber 34 (measurement device), a load lock 35 and a glove box 36.

Though the preparation device that prepares a sample and the measurement device that measures the sample are integrally configured with each other in the present embodiment, the preparation device and the measurement device may be configured separately, not integrally.

The transfer device 30 is transfer means for transferring a sample substrate under vacuum and is a transfer robot provided with a robot arm therein. The transfer device 30 is located at the center of the preparation evaluation unit 3 when viewed from above, and the deposition chambers 31, 32, 33, the measurement chamber 34, the load lock 35 and the glove box 36 are disposed around the transfer device 30.

The load lock 35 is an auxiliary vacuum chamber and is a stage on which a sample substrate is placed. The transfer device 30 takes out a sample substrate placed on the load lock 35 and transfers it to the deposition chambers 31, 32, 33.

The deposition chambers 31, 32, 33 are each a vacuum chamber for performing deposition, hit source materials with an argon (Ar) gas of high energy to cause the materials to scatter and deposit on a sample substrate. Specifically, Ar gas and atmospheric gas (O₂, N₂, H₂ and the like) are enclosed in the deposition chamber 31, 32, 33 to cause plasma discharge between a material sintered body called a target and a substrate. Here, Ar ions accelerated by voltage applied across the electrodes collide with the target to cause the material to scatter and deposit on the substrate. The transfer device 30 transfers the deposited sample to the measurement chamber 34.

The measurement chamber 34 is provided with a prober stage for measuring electrical conductivity of the sample and automatically measures the ion conductivity of the thin film formed on the substrate. The measurement chamber 34 is further provided with a light emitting device and a light-receiving sensor for measuring optical reflectivity of the thin film, which allows measurements of the thickness of the thin film at the same time. After completion of measurement in the measurement chamber 34, the transfer device 30 returns the sample to the load lock 35 for storing.

The glove box 36 is a closed vessel closed to the atmosphere where a worker engineers a sample.

Though the glove box 36 is disposed for performing deposition of semiconductor materials in the present embodiment, the glove box 36 is not an indispensable element for the preparation evaluation unit 3.

Though a sputtering method is employed as a deposition method in the present embodiment, a pulse laser deposition (PLD) method, a chemical vapor deposition (CVD) method or the like may be employed. Furthermore, the physical property value to be measured may be, for example, a band gap, a relative dielectric constant or the like without being limited to ion conductivity. As such, the deposition chambers 31, 32, 33 and the measurement chamber 34 are mere examples of preparation means (preparation device) and measurement means (measurement device) of a sample and can appropriately be changed depending on the preparation method of a sample and the purpose of an experiment (the physical property to be optimized).

Additionally, the sample to be prepared is may be, for example, a polymeric material, a metal structure material or the like without being limited to a semiconductor material. In the case where a metal structure material is prepared, for example, optimization is performed by measuring the mechanical strength or the like. In addition, pharmaceutical products, biological substances and foods other than material substances may be employed as subjects.

Furthermore, the sample to be prepared may be applied to evaluation for a mode including liquid, solid and gas and their mixed states of, for example, an organic compound, an inorganic compound and a mixture thereof without being limited to the use for a thin-film material. Hence, the sample (substance) prepared in the present system is not limited to a particular one.

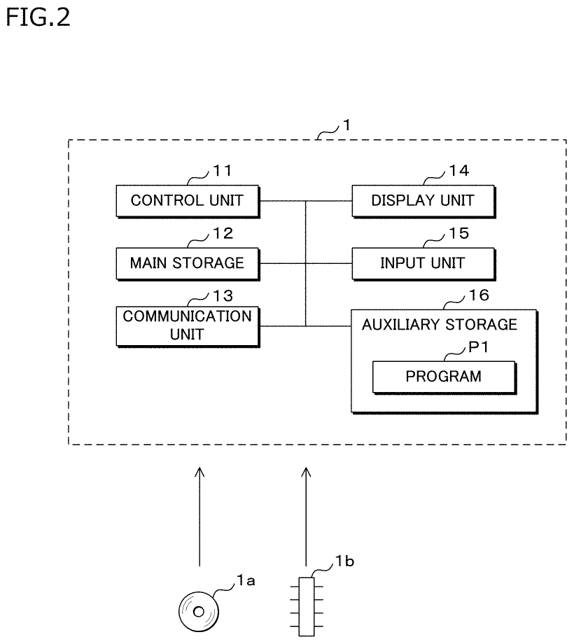

FIG. 2 is a block diagram showing an example of the configuration of the prediction device 1. The prediction device 1 is provided with a control unit 11, a main storage 12, a communication unit 13, a display unit 14, an input unit 15 and an auxiliary storage 16.

The control unit 11 has one or more arithmetic processing unit such as a CPU (Central Processing Unit), an MPU (Micro-Processing Unit), a GPU (Graphics Processing Unit) or the like and performs various kinds of information processing, control processing and the like by reading and executing a program P1 stored in the auxiliary storage 16. The main storage 12 is a temporary storage area such as a RAM (Random Access Memory), and temporarily stores data necessary for the control unit 11 to execute arithmetic processing. The communication unit 13 is a communication module for performing processing related to communication, and transmits and receives information to/from the outside. The display unit 14 is a display device such as a liquid crystal display, and displays an image provided by the control unit 11. The input unit 15 is an operation interface such as a keyboard, a mouse and the like and accepts an operation input from the user. The auxiliary storage 16 is a nonvolatile storage area such as a hard disk or the like, and stores the program P1 necessary for the control unit 11 to execute processing and other data.

Note that the prediction device 1 may be provided with a reading unit that reads a portable storage medium 1a such as a CD (Compact Disk)-ROM or a DVD (Digital Versatile Disc)-ROM, and read the program P1 from the portable storage medium 1a and execute it. Alternatively, the prediction device 1 may read the program P1 from a semiconductor memory 1b.

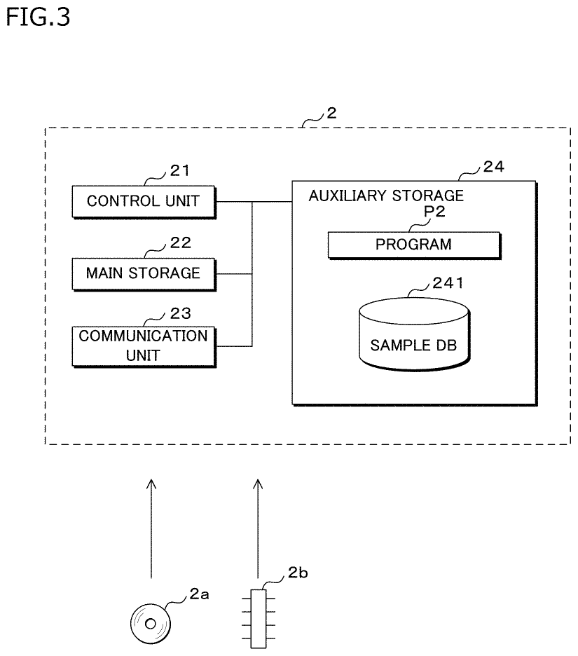

FIG. 3 is a block diagram showing an example of the configuration of the server 2. The server 2 is provided with a control unit 21, a main storage 22, a communication unit 23 and an auxiliary storage 24.

The control unit 21 has one or more arithmetic processing unit such as a CPU or the like, and performs various kinds of information processing, control processing and the like by reading a program P2 stored in the auxiliary storage 24 and executing it. The main storage 22 is a temporary storage area such as a RAM or the like and temporarily stores data necessary for the control unit 21 to execute arithmetic processing. The communication unit 23 is a communication module for performing processing related to communication, and transmits and receives information to/from the outside.

The auxiliary storage 24 is a nonvolatile storage area such as a hard disk or the like, and stores the program P2 necessary for the control unit 21 to execute processing and other data. Furthermore, the auxiliary storage 24 stores a sample DB 241. The sample DB 241 is a database storing information on samples prepared by the preparation evaluation unit 3.

It is noted that the auxiliary storage 24 may be an external storage device connected to the server 2. Furthermore, the server 2 may be a multicomputer formed of multiple computers or may be a virtual machine virtually constructed by software.

Moreover, the server 2 may be provided with a reading unit that reads a portable storage medium 2a such as a CD (Compact Disk)-ROM or a DVD (Digital Versatile Disc)-ROM, and read the program P2 from the portable storage medium 2a and execute it. Alternatively, the server 2 may read the program P2 from a semiconductor memory 2b.

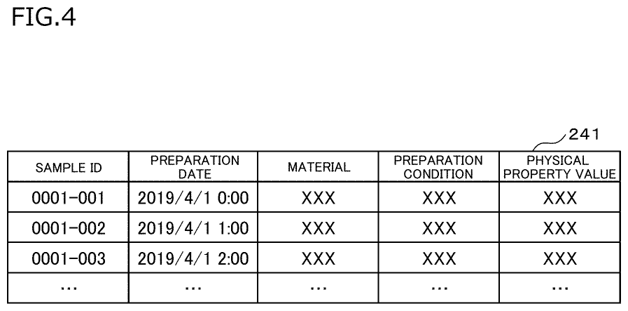

FIG. 4 is an explanatory view showing an example of a record layout of the sample DB 241. The sample DB 241 includes a sample ID column, a preparation date column, a material column, a preparation condition column and a physical property value column. The sample ID column stores sample IDs for identifying samples prepared by the preparation evaluation unit 3. The preparation date column, the material column, the preparation condition column and the physical property value column respectively store the date and time when a sample is prepared, the material (element), the preparation condition and the physical property value of the sample in association with a sample ID.

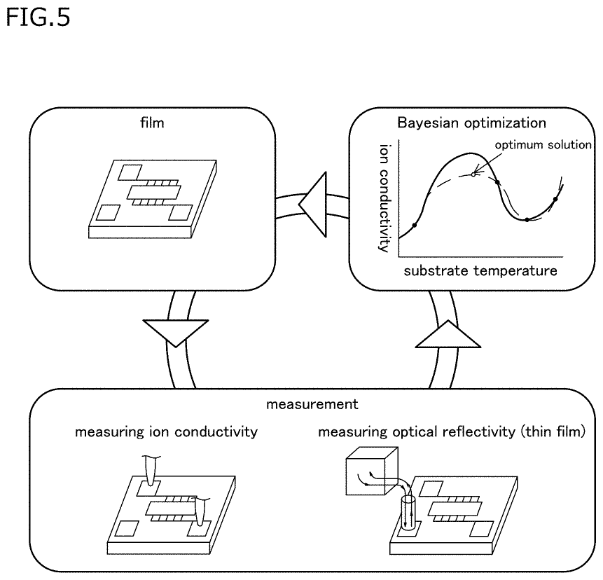

FIG. 5 is an explanatory view showing an outline of Embodiment 1. FIG. 5 conceptually shows a state in which preparation of a sample and measurement of a physical property value of the sample by the preparation evaluation unit 3 and a prediction of an optimum sample by the prediction device 1 are sequentially repeated. Based on FIG. 5, the outline of the present embodiment will be described.

As already described, the prediction device 1 performs processing of predicting a preparation condition of a sample that optimizes the physical property value from a data set including the preparation conditions and physical property values of multiple samples. In the present embodiment, sequential searches are performed using a method of Bayesian optimization.

Bayesian optimization is a method used, in a data set of an objective variable y and an n-dimensional explanation variable xi (i=1, 2, . . . n), to search for the explanation variable xi that maximizes (or minimizes) the objective variable y assuming that the function of y=f(xi) follows a Gaussian process if the function ƒ is unknown. The unknown function ƒ is assumed to follow the Gaussian process, which can highly optimize various types of objective variables y with simpler processing in comparison with the case where an objective variable y and an explanation variable xi are assumed to follow another distribution.

Note that Bayesian optimization itself is a known method, and thus the detailed description thereof will not be made in the present embodiment.

In addition, though Bayesian optimization is employed as a method of sequentially searching an optimum sample in the present embodiment, the prediction device 1 is only required to be able to search for an optimum solution from a data set sequentially added and may thus employ any method such as reinforcement learning, linear search or the like.

In the present embodiment, it is assumed that the objective variable y corresponds to a physical property value to be measured in the measurement chamber 34, and the explanation variable xi corresponds to various types of parameters that define a preparation condition for a sample. More specifically, it is assumed that the objective variable y is ion conductivity (S/m) of a thin-film sample while the explanation variable xi is a substrate temperature (° C.), a partial pressure of hydrogen (Pa), a partial pressure of nitrogen (Pa), a deposition rate (A/s) at the time of preparing a sample or the like. Note that these parameters are all illustrative, and any parameters may be possible as long as the objective variable defines a physical property value while the explanation variable defines the preparation condition.

For example, the prediction device 1 first randomly generates preparation conditions of a predetermined number of samples and inputs the conditions to the preparation evaluation unit 3 to instruct the preparation evaluation unit 3 to prepare a sample. When a preparation condition is input from the prediction device 1, the transfer device 30 of the preparation evaluation unit 3 takes out a sample substrate previously set in the load lock 35 and transfers the substrate to any one of the deposition chambers 31, 32, 33. The preparation evaluation unit 3 prepares (deposits) a sample in the deposition chamber 31, 32, 33 according to the preparation condition.

After completion of preparation of a sample, the transfer device 30 takes out the sample from the deposition chamber 31, 32, 33 and transfers the sample to the measurement chamber 34. The preparation evaluation unit 3 measures the physical property value of the sample in the measurement chamber 34. After completion of measurement of the physical property value, the transfer device 30 takes out the sample from the measurement chamber 34 and transfers it to the load lock 35 for storing.

The preparation evaluation unit 3 repeats the above-described processing to prepare samples under the preparation conditions randomly defined and measures their physical property values. This makes it possible to generate a data set including the preparation conditions and the physical property values of the predetermined number of samples.

Though samples are prepared under randomly defined preparation conditions in order to generate a data set in the present embodiment, a data set manually prepared may be employed as an initial data set. Furthermore, the information on the data set accumulated in the past is utilized and subjected to principal component analysis, sparse modeling, or the like to obtain information. The obtained information may be employed as an initial data set. Input of appropriate initial values enables convergence with a small number of trials, which enables a search for an optimum sample more suitably.

The prediction device 1 performs Bayesian optimization based on this data set to predict the preparation condition of a sample that optimizes the physical property value. At the upper right of FIG. 5, the contents of the processing of Bayesian optimization is shown by a conceptual graph. The horizontal axis represents an explanation variable (substrate temperature), and the vertical axis represents an objective variable (ion conductivity). Note that though a search is actually made in a multidimensional space such as a three-dimensional or more, FIG. 5 shows a two-dimensional graph for the convenience's sake.

Assuming that the data set follows Gaussian distribution, the prediction device 1 searches for a point with the maximum physical property value. That is, the prediction device 1 predicts the average and variance from distribution for the data set and draws a Gaussian process. The prediction device 1 performs optimization using a function for evaluation called an acquisition function to search for a point with the maximum physical property value.

It is noted that the prediction device 1 may previously accept a setting input of setting a numerical range for the preparation condition (explanation variable) and a numerical range for the physical property value (objective variable) and may perform a search within the numerical range.

According to the above-described processing, the prediction device 1 predicts the explanation variable xi that appears to be optimum, that is, a preparation condition of an optimum sample. The prediction device 1 inputs the predicted preparation condition to the preparation evaluation unit 3. The preparation evaluation unit 3 transfers, prepares and measures a sample as in the preparation of the initial sample and returns a physical property value of the prepared sample to the prediction device 1.

The prediction device 1 adds the preparation condition of the sample input as above and the physical property value of this sample to the data set as new sample information and predicts a preparation condition that appears to be optimum again. More specifically, the prediction device 1 predicts the average and variance of the new data set to update the acquisition function. The prediction device 1 searches for an optimum point from the updated acquisition function again to draw a preparation condition of a sample to be prepared next. Hence, the prediction device 1 sequentially predicts the preparation condition of a sample that appears to be optimum at the (k+1)th time based on the data set of the samples prepared before the k-th time and instructs the preparation evaluation unit 3 to prepare and measure a sample.

For example, the prediction device 1 converges a series of processing (experiments) if the data set sequentially added satisfies a predetermined convergence condition. For example, in the case where the current number of searches is the (k+1)th time, the prediction device 1 compares the sample information at the (k+1)th time and the sample information at the k-th time and determines whether or not the physical property value is converged to a local solution.

More specifically, the prediction device 1 determines whether or not the sequentially-predicted preparation condition of samples is the same preparation condition consecutively for a predetermined number of times (multiple times). Note that “the same” includes substantially the same case where parameters approximate to each other in a predetermined numerical range as well as the case of a completely identical parameter.

Furthermore, the prediction device 1 determines whether or not the physical property value (measurement value) measured by the preparation evaluation unit 3 is more proper than the prediction value of the physical property value predicted under the preparation condition input to the preparation evaluation unit 3. That is, the prediction device 1 determines whether or not the actual physical property value is superior to the physical property value (objective variable) predicted together with the preparation condition (explanation variable) by Bayesian optimization. If the physical property value is to be maximized, it is determined whether or not the actual physical property value is equal to or larger than the prediction value. In contrast, if the physical property value is to be minimized, it is determined whether or not the actual physical property value is equal to or less than the prediction value.

As a result of combining the above-mentioned two determination conditions, if the same preparation condition is predicted the consecutive multiple number of times and the measured physical property value is more proper than the prediction value, the prediction device 1 determines that the physical property value is fully converged and ends the processing.

Though the convergence of the physical property value is regarded as a convergence condition in the above description, the prediction device 1 may preset the upper limit of the number of searches, for example, and end the processing if the upper limit is reached.

After completion of the processing, the prediction device 1 displays the sample information of the samples held as data set and the sample information of an optimum sample whose physical property value is an optimum value (maximum value or minimum value). In addition, the prediction device 1 outputs the data set to the server 2 and stores it in the sample DB 241.

Though not especially described in the above, the prediction device 1 may place various constraints depending on the environment of the experiments when performing Bayesian optimization.

For example, the prediction device 1 places a constraint depending on the condition of a preparable sample in the preparation evaluation unit 3, that is, the preparation capability. More specifically, the prediction device 1 places a numerical range (upper limit and lower limit) settable in the preparation evaluation unit 3 as constraints for parameters such as a substrate temperature, a partial pressure of hydrogen, a partial pressure of nitrogen and the like. In the case where the substrate temperature, the partial pressure of hydrogen, the partial pressure of nitrogen and the like are each settable only with a discrete numerical interval (settable only with an interval of numerical values of 100° C., 110° C. . . . , for example), the numerical interval is set as a constraint. When performing Bayesian optimization, the prediction device 1 searches for the preparation condition of an optimum sample according to these constraints (preparation capability). The provision of the constraints depending on the preparation capability in the preparation evaluation unit 3 enables more suitable search for an optimum sample.

Hence, according to the present embodiment, it is possible to prepare a sample and measure and optimize the physical property value thereof in a fully automated cycle. This makes it possible to accumulate a variety of and a vast number of experimental results and suitably support a researcher or the like at the same time.

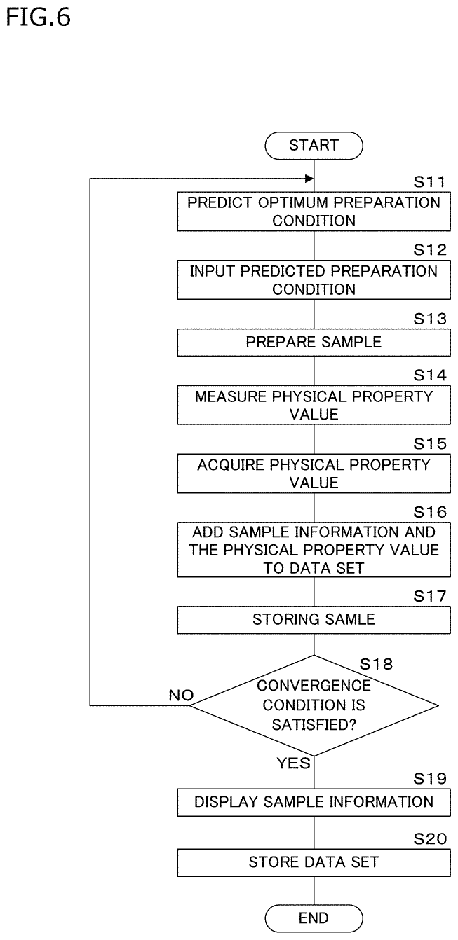

FIG. 6 is a flowchart showing an example of the processing procedure executed by the prediction device 1. With reference to FIG. 6, the contents of the processing to be executed by the prediction device 1 will be described. Note that the following description will be made assuming that the prediction device 1 already holds a data set including a predetermined number of samples.

The control unit 11 of the prediction device 1 predicts a preparation condition of a sample that optimizes the physical property value based on the data set including preparation conditions of samples and physical property values of the samples (substance information) (step S11). More specifically, the control unit 11 performs Bayesian optimization regarding the preparation condition as an explanation variable and the physical property value as an objective variable to predict a preparation condition of a sample that maximizes (or minimizes) the physical property value as described above.

The control unit 11 inputs the predicted preparation condition to the preparation evaluation unit 3 (step S12). Then, the control unit 11 causes the transfer device 30 to transfer a sample substrate from the load lock 35 to the deposition chamber 31, 32, 33 to prepare a sample under the input preparation condition therein (step S13).

In the case where the preparation of the sample is completed, the control unit 11 causes the transfer device 30 to transfer the sample from the deposition chamber 31, 32, 33 to the measurement chamber 34 so as to measure a physical property value of the sample therein (step S14). The control unit 11 acquires the physical property value measured at step S14 from the preparation evaluation unit 3 (step S15). The control unit 11 adds sample information indicating the preparation condition input at step S12 and the physical property value acquired at step S15 to the data set (step S16). In the case where addition to the data set is completed, the control unit 11 transfers the sample from the measurement chamber 34 to the load lock 35 for storing (step S17).

The control unit 11 determines whether or not a predetermined convergence condition is satisfied with reference to the data set (step S18). If determining that the convergence condition is not satisfied (S18: NO), the control unit 11 returns the processing to step S11. If determining that the convergence condition is satisfied (S18: YES), the control unit 11 displays the sample information of the samples held as a data set and the sample information of an optimum sample whose physical property value is an optimum value (step S19). Additionally, the control unit 11 outputs the data set to the server 2 so as to store the data set in the sample DB 241 (step S20). The control unit 11 ends the series of processing.

Though the objective variable for optimization is regarded as a physical property value in the present embodiment, the objective variable may be structure information representing the structure of a sample (crystal structure or the like) without being limited to the physical property value. The structure information includes information on, for example, symmetry and a lattice constant of a crystal, vibrational modes of atoms derived from a local internal structure and molecular structure of a crystal and the like. In this case, in the measurement chamber 34 (the measurement device) of the preparation evaluation unit, an X-ray diffraction pattern, Raman spectrum and the like are measured, and the above-mentioned structure information is drawn from observation amounts (measurement values) such as a diffraction peak position and an intensity ratio obtained from X-ray diffraction and a scattering peak position and a half value width obtained from Raman spectroscopy. The prediction device 1 performs sequential searches for an optimum sample regarding the derived structure information as an objective variable. It is thus sufficient for the prediction device 1 to only be able to measure and optimize substance information representing the physical property or the structure of a sample (substance), and the parameter to be measured is not limited to the physical property value.

As such, according to Embodiment 1, it is possible to prepare a sample and measure and optimize the physical property value (substance information) thereof in a fully automated cycle, which enables a suitable implement of materials informatics.

Furthermore, according to Embodiment 1, use of a method of Bayesian optimization enables optimization of a sample with simple processing, which can reduce the computational complexity (the number of searches) performed by the prediction device 1.

Moreover, according to Embodiment 1, provision of constraints depending on the preparation capability of the preparation evaluation unit 3 enables suitable optimization of a sample taking the environment of the experiment into account.

### Embodiment 2

In the present embodiment, a mode in which multiple physical property values can simultaneously be measured will be described. Contents overlapping those of Embodiment 1 are denoted by the same reference signs and descriptions thereof are not made.

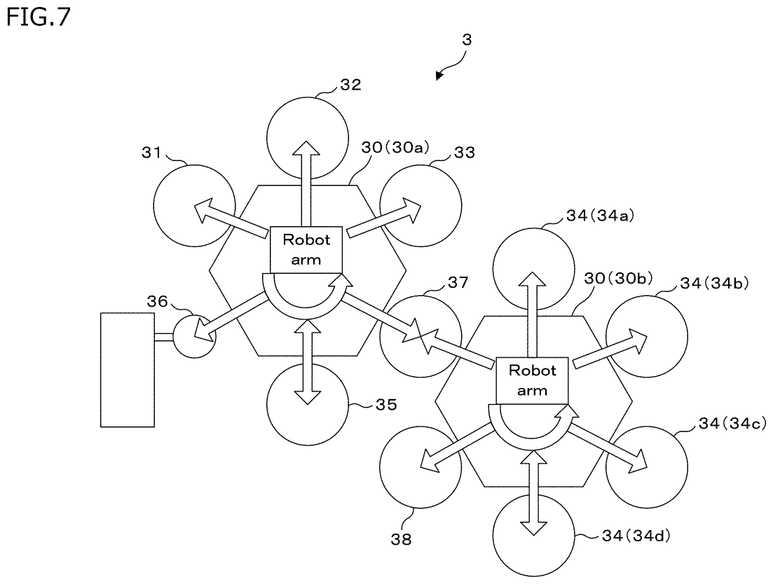

FIG. 7 is a schematic view showing an example of the configuration of a preparation evaluation unit 3 according to Embodiment 2. The preparation evaluation unit 3 according to the present embodiment is configured to include two transfer devices 30, 30 around which deposition chambers 31, 32, 33, a measurement chamber 34 and the like are disposed.

More specifically, the preparation evaluation unit 3 is provided with a first transfer device 30a and a second transfer device 30b. The first transfer device 30a is connected to the deposition chambers 31, 32, 33, a load lock 35 and a glove box 36. Furthermore, the first transfer device 30a and the second transfer device 30b are connected to each other via a delivery chamber 37. The delivery chamber 37 is an auxiliary vacuum chamber as in the glove box 36, for example, and is a stage for delivering a sample from the first transfer device 30a to the second transfer device 30b.

The second transfer device 30b is connected to multiple measurement chambers 34a, 34b, 34c, 34d and an ejection chamber 38. The measurement chambers 34a, 34b, 34c, 34d are measurement chambers for measuring different types of physical property values from one another. The physical property values measured at the respective measurement chambers 34a, 34b, 34c, 34d are not particularly limited but includes, for example, ion conductivity, a band gap (eV), a relative dielectric constant, thermal conductivity (W/m K). The ejection chamber 38 ejects a sample after measurement to the outside of the device.

Note that the preparation evaluation unit 3 may preferably measure the crystal structure and the molecular structure of a sample, not the physical property value, of a sample at any one of the measurement chambers 34a, 34b, 34c, 34d. For example, the preparation evaluation unit 3 measures an X-ray diffraction pattern, Raman spectra and the like. This allows the preparation evaluation unit 3 to acquire the structure information of an optimum sample and accumulate the structure information of each of the prepared samples in the sample DB 241 as well.

The preparation evaluation unit 3 according to the present embodiment prepares a sample on the first transfer device 30a side and measures the physical property value thereof on the second transfer device 30b side. That is, the first transfer device 30a transfers a sample substrate from the load lock 35 to the deposition chambers 31, 32, 33 to prepare (deposit) a sample therein according to the preparation condition input from the prediction device 1. Then, the first transfer device 30a transfers the sample to the delivery chamber 37. The second transfer device 30b takes out the sample from the delivery chamber 37 and transfers the sample to the measurement chambers 34a, 34b, 34c, 34d one after another to measure the multiple types of the physical property values. After completion of the measurements, the second transfer device 30b transfers the sample to the ejection chamber 38 to eject the sample to the outside (housing tray for a sample substrate, for example).

It is preferable that the ejection chamber 38 is configured not only to eject a sample to the outside but also to mark a sample substrate with a sample ID (identifying information) that can uniquely identify samples prepared one after another. Though a marking method is not limited to a particular one, marking is performed by a laser marker, for example. For example, the preparation evaluation unit 3 is equipped with an emission device that emits laser light in the ejection chamber 38 and controls the emission device to print a sample ID on a substrate. In the case where samples are prepared and measured by the preparation evaluation unit 3 without human intervention, the worker can easily identify the samples indicated by a data set upon confirmation of the samples thereafter.

Though the marking details include printing a sample ID (character string) as described, it may include printing a bar code such as QR Code (registered trademark), for example.

As described above, provision of the multiple measurement chambers 34a, 34b, 34c and 34 allows the preparation evaluation unit 3 to measure multiple physical property values for one sample. The prediction device 1 performs optimization of a sample regarding one or more physical property values as objective variables out of multiple physical property values that can be measured by the preparation evaluation unit 3. For example, at a start of a series of processing, the prediction device 1 accepts a selection input of selecting physical property values as objective variables from the worker. The prediction device 1 performs Bayesian optimization regarding one or more physical property values arbitrarily selected as objective variables.

Note that in the case where multiple physical property values are selected as objective variables, it is preferable that the prediction device 1 accepts a setting input of priorities indicating the degree of prioritizing optimization for the respective physical property values and assigns weights to them. For example, the prediction device 1 accepts an input of a hyper parameter (for example, a value between 0 and 1, wa, wb, wc . . . ) representing weights of the priorities for the multiple types of the physical property values. Assuming that the physical property value of the physical property A is a and the physical property value of the physical property B is b, a figure of merit is defined as wa*a*wb*b, whereby optimization is performed so as to maximize the figure of merit. When performing Bayesian optimization, the prediction device 1 calculates a figure of merit according to the input hyper parameter and predicts a preparation method of an optimum sample. This makes it possible to suitably evaluate each of the physical property values when optimization is performed employing multiple physical property values in combination.

As in the processing in Embodiment 1, in the following processing, the prediction device 1 performs sequential searches from the preparation conditions and the physical property values of samples sequentially prepared and measured to predict a preparation condition of an optimum sample. The prediction device 1 sequentially inputs the predicted preparation condition to the preparation evaluation unit 3 to prepare samples therein. In the case where a predetermined convergence condition is satisfied, the prediction device 1 ends the series of processing and stores in the sample DB 241 the data set including the structure information as well as the preparation conditions and physical property values of the prepared samples.

Note that in the above-described processing, the preparation evaluation unit 3 is preferably configured to measure all the measurable physical property values regardless of the selected physical property value (objective variable). This makes it possible to accumulate in the sample DB 241 the measurement results for a large number of physical property values with single processing regardless of the purpose of the experiment (the physical property to be optimized).

As such, according to the present embodiment, a single preparation evaluation unit 3 can measure multiple physical property values to optimize a sample. Since the present embodiment is common to Embodiment 1 except for the above description, flowcharts and other detailed descriptions are not made in the present embodiment.

### Embodiment 3

Described in Embodiment 1 is a configuration in which the prediction device 1 and the preparation evaluation unit 3 prepare a sample, and measure and optimize the physical property value. Described in Embodiment 2 is a configuration in which multiple physical property values can be measured. Described in Embodiment 3 is a configuration in which the above-mentioned embodiments are combined, and the preparation evaluation system is made available by an external user.

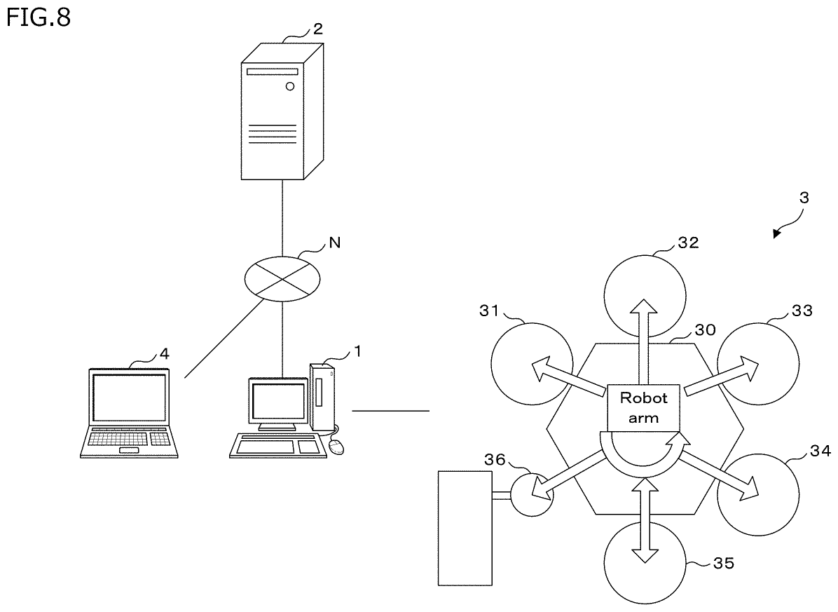

FIG. 8 is a schematic view showing an example of the configuration of a preparation evaluation system according to Embodiment 3. The preparation evaluation system according to the present embodiment is connected to a terminal 4 via a network N. The network 4 is a terminal device of the external user and is configured so as to communicate with a server 2.

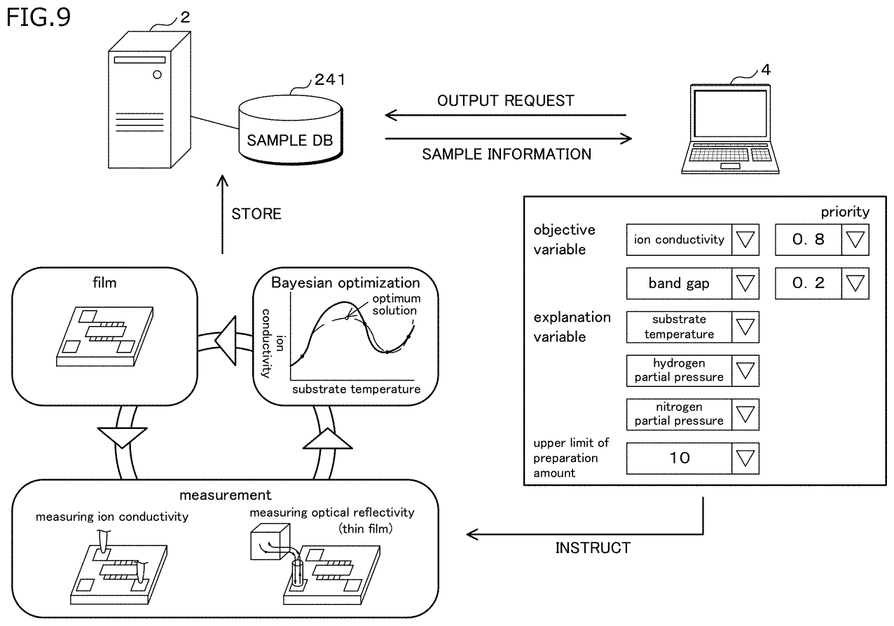

FIG. 9 is an explanatory view showing an outline of Embodiment 3. FIG. 9 conceptually shows a situation in which the terminal 4 acquires sample information stored in the sample DB 241 from the server 2, and the prediction device 1 and the preparation evaluation unit 3 prepare, measure and optimize a sample according to a condition set by the user.

In the present embodiment, the server 2 provides an application programmable interface (API) service that makes the sample information stored in the sample DB 241 available to the external user. For example, the server 2 accepts an output request of any sample information from the terminal 4 in response to an operation input performed on a predetermined Web browser and outputs the requested sample information (material, preparation condition, physical property value, structure information of a sample and the like) to the terminal 4. For example, the server 2 offers a database search engine regarding various parameters such as a physical property value, a preparation condition and the like as search queries and searches for desired sample information from the sample DB 241 according to the parameters input through the terminal 4.

It is preferable that when outputting the sample information to the terminal 4, the server 2 not only outputs sample information (raw data) stored in the sample DB 241 but also calculates to what extent each of the parameters (explanation variable) defined as a preparation condition at a time of preparing a sample is correlated with the physical property value (objective variable), that is, the degree of correlation of the preparation condition with the physical property value and presents it to the user. For example, the server 2 calculates for each parameter the degree of correlation of the preparation condition with the physical property value by means of sparse modeling, regression analysis, principal component analysis or the like from the data set stored in the sample DB 241. This makes it possible to provide information useful for the user such as to what extent each of the parameters of the preparation condition contributes to the physical property of a sample.

In the present embodiment, the server 2 not only provides sample information but also accepts a request for processing relating to preparation of a sample and measurement and optimization of the physical property value from the external user and operates the prediction device 1 and the preparation evaluation unit 3. That is, the server 2 accepts a request for an experiment from the external user and conducts experiments in the prediction device 1 and the preparation evaluation unit 3.

More specifically, the server 2 accepts from the terminal 4 setting inputs of the types of parameters for the preparation condition regarded as the explanation variables and the types of a physical property value regarded as the objective variables as well as the material (element) of a sample to be prepared. At the right side of FIG. 9, a screen image displayed on the terminal 4 at the time of performing a setting input is shown. The server 2 accepts a selection input of selecting the types of the parameters for the preparation condition regarded as the explanation variables upon Bayesian optimization and the types of the physical property values measured as the objective variables. In this case, it is preferable that the server 2 accepts a setting input of priorities for the physical property values as described in Embodiment 2. The server 2 executes Bayesian optimization based on the set parameters.

Note that though the hyper parameter used for Bayesian optimization processing includes acceptance of a setting input of priorities for the physical property values in the above description, a setting input for another hyper parameter may be possible.

In addition, it is preferable that upon the setting input described above, the server 2 accepts a setting input of an upper limit of the preparation amount of a sample. The preparation amount of a sample includes, for example, the number of preparations (the number of searches), a time required for preparing a sample through the entire experiment (experiment time) or the like. When performing sequential searches for an optimum sample, the prediction device 1 determines whether or not the processing is to be ended based on the upper limit arbitrarily set by the external user. This allows the external user to control a total experimental schedule.

Though examples of the upper limit of the preparation amount include the number of prepared samples and a preparation time as described above, the server 2 may accept a setting input of a budget that can actually be invested in the experiment as an upper limit of the preparation amount, for example. In this case, the server 2 calculates the number of samples that can be prepared from the upper limit of the budget based on the material price per unit of pieces and sets the number as a convergence condition, for example. Alternatively, the server 2 may set a cost per unit time from the running cost spent for calculation in the prediction device 1 and calculate the upper limit of the preparation amount. As such, the preparation amount of a sample regarded as a convergence condition is not limited to a physical parameter such as the number of prepared samples, a preparation time or the like, and any parameters that can be referred to when an experiment is planned may be possible.

The server 2 outputs setting contents in the terminal 4 to the prediction device 1 and instructs the prediction device 1 to perform sequential searches for an optimum sample in conjunction with the preparation evaluation unit 3. Though the prediction device 1 here may start the processing after randomly generating preparation conditions as in Embodiment 1, it may preferably start the processing after accepting from the external user a setting input of setting an initial value of the data set used for predicting an optimum sample with reference to the sample information stored (accumulated) in the sample DB 241.

For example, the sever 2 accepts from the terminal 4 a selection input of selecting any sample information from the sample DB 241 and sets the sample information selected by the user to the initial value of the data set. That is, as described above, the server 2 accepts a setting input of setting the sample information output in response to an output request from the terminal 4 to the initial value of the data set as described above and outputs the sample information to the prediction device 1. Furthermore, the server 2 outputs other setting contents (preparation condition regarded as the explanation variable, physical property value regarded as the objective variable, priorities, upper limit of the preparation amount, and the like) set as described above to the prediction device 1 and instructs it to start the processing.

Alternatively, the server 2 may utilize information obtained by performing a method such as principal component analysis, sparse modeling or the like on the sample information accumulated in the sample DB 241, not the sample information accumulated in the sample DB 241 as it is, as the initial value of the data set. That is, the server 2 extracts the sample information from the sample DB 241 depending on the material, the explanation variable (the types of parameters for the preparation condition) and the objective variable (the types of physical property value) that are set by the user, performs a simulation to predict the preparation conditions and the physical property values that are optimum for a sample as the initial value of a data set and generates an initial data set. Hence, the server 2 may not only utilize the sample information stored in the sample DB 241 as it is but also set a simulation result based on the sample information in the past as the initial value of a data set.

Through the above-mentioned processing, an infinite number of users can turn the experimental cycle by suitably utilizing the sample information accumulated in the sample DB 241.

In the case where the convergence condition is satisfied to complete the processing in the prediction device 1 and the preparation evaluation unit 3, the server 2 acquires a data set indicating the sample information of the prepared samples from the prediction device 1. The server 2 stores the acquired data set in the sample DB 241 and outputs the sample information of the samples included in the data set to the terminal 4 as a request source.

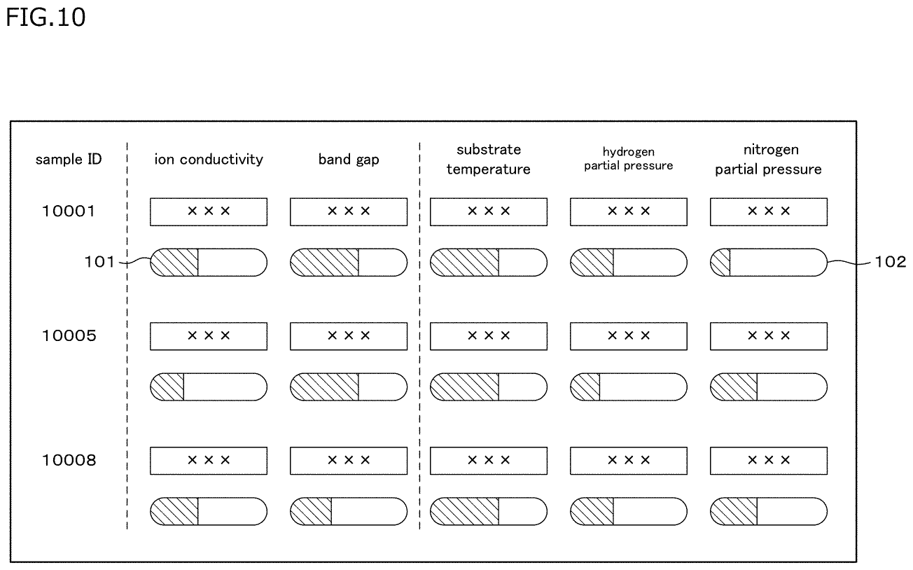

FIG. 10 is an explanatory view showing an example of a display screen of sample information. The terminal 4 displays a screen in FIG. 10 based on an output from the server 2, for example.

For example, the terminal 4 displays in a list the physical property value of samples and parameters included in the preparation condition that are associated with sample IDs (identifying information) attached to the respective samples. Note that pieces of information on the samples are displayed in the order of approximation of the physical property value to the optimum value, the first being the most approximate. In the case where multiple physical property values are measured as the objective variables, the degree of approximation may be decided by calculating Euclidean distance based on, for example, the measurement values of the physical property values obtained by measuring a sample and optimum values of the physical property values searched by Bayesian optimization.

The terminal 4 displays physical property values included in a data set and parameters included in the preparation condition for each sample. As shown in FIG. 10, for example, the physical property values and the preparation conditions are displayed by numerical values. Moreover, as to the physical property values, a matching display bar 101 indicating the degree of approximation to an optimum value is displayed for each physical property value. For example, as to the each of the physical property values, the server 2 calculates the difference between an optimum value and a measurement value, normalizes the difference, and displays the matching display bar 101 depending on the normalized difference, that is, the degree of approximation to the optimum value.

In addition, as to the preparation condition, for example, the server calculates the degree of correlation with the physical property value for each parameter and displays a correlation degree display bar 102 depending on the degree of correlation. Thus, the terminal 4 displays the sample information of the samples included in the data set and presents the degree of optimization (the degree of approximation) of the physical property value, the degree of correlation for each of the parameters included in the preparation condition and the like for the user's information.

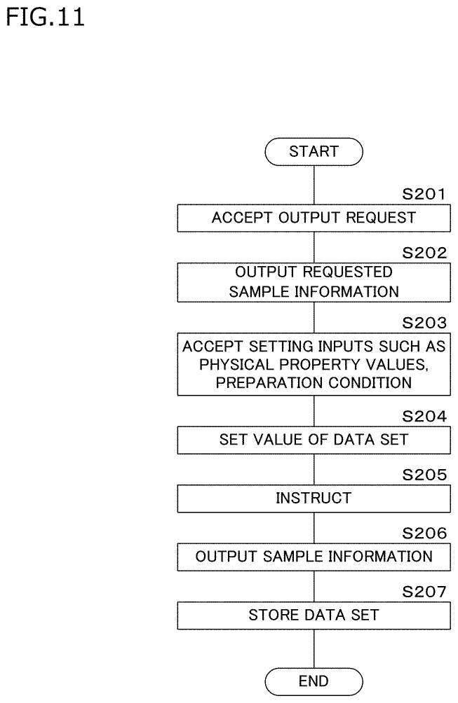

FIG. 11 is a flowchart showing an example of the processing procedure to be executed by the server 2. With reference to FIG. 11, contents of the processing to be executed by the server 2 according to Embodiment 3 will be described.

The control unit 21 of the server 2 accepts an output request of any sample information stored in the sample DB 241 from the terminal 4 of the external user (step S201). The control unit 21 outputs the requested sample information to the terminal 4 (step S202). In this case, the control unit 21 calculates the degree of correlation of the preparation condition with the physical property value for each of the parameters included in the preparation condition from the requested data set at a time of preparing the sample, and outputs the calculated the degree of correlation.

The control unit 21 accepts various setting inputs such as the types of the physical property values as the objective variables, the types of the parameters in the preparation condition as the explanation variables, the priorities of the physical property values, an upper limit of the preparation amount of samples as a convergence condition and the like (step S203). Furthermore, the control unit 21 accepts a setting input of setting the initial value of the data set with reference to the sample information stored in the sample DB 241 (step S204). For example, the control unit 21 accepts a setting input of setting the sample information output at step S202 to the initial value of a data set. Alternatively, the control unit 21 may perform a simulation (for example, principal component analysis) based on the sample information stored in the sample DB 241 depending on the contents of the settings such as the object variable and the explanation variable set at step S203 to generate an initial data set.

The control unit 21 outputs the contents of the setting that are set at step S203 and the data set that is set at step S204 to the prediction device 1 and instructs the prediction device 1 to perform sequential predictions of an optimum sample in conjunction with the preparation evaluation unit 3 (step S205).

In the case where the processing in the prediction device 1 and the preparation evaluation unit 3 is completed, the control unit 21 acquires a data set including the preparation conditions and the physical property values of the sample prepared in the preparation evaluation unit 3 from the prediction device 1 and outputs the sample information to the terminal 4 (step S206). For example, the server 2 displays the sample information of the samples prepared by the preparation evaluation unit 3 in association with the sample IDs (identifying information) in a list. In this case, for example, the server 2 displays in a list the sample information in the order of the degree of approximation between the measurement value of the physical property value measured by the preparation evaluation unit 3 and the optimum value searched by Bayesian optimization. Additionally, the server 2 displays the degree of approximation to the optimum value for each of the preparation conditions by the matching display bar 101 and the degree of correlation with the physical property value for each of the parameters included in the preparation condition by the correlation degree display bar 102, for example. The control unit 21 stores the data set in the sample DB 241 (step S207) and ends the series of processing.

As such, according to Embodiment 3, the external user can use the sample information accumulated in the sample DB 241 and suitably implement materials informatics by utilizing the present system.

### Embodiment 4

In the present embodiment, experimental data obtained by using the above-described preparation evaluation system will be described.

The inventors of the present application conducted an experiment of minimizing the electrical resistance of an Nb doped TiO₂ thin film deposited on a glass substrate using the above-described preparation evaluation system. A sputtering method was employed for deposition, and two different types of Ti_{0.94}Nb_{0.06}O₂ and Ti_{1.98}Nb_{0.02}O₃ that are different in the ratio of Ti and O were employed for targets. By adjusting the mixture ratio between an Ar gas and the gas mixture of Ar (99%) and O₂ (1%), an oxygen content (oxygen partial pressure) in a thin film with the minimum electrical resistance was searched. Thin films were deposited using magnetron sputtering, one of a physical vapor deposition technique, with total pressure of 0.50 Pa and the output of an RF power source of 100 W. The deposition time and the maximum thickness of the thin film were respectively one hour and 120 nm. The substrate temperature during the deposition was room temperature, and annealing treatment at 400° C. for 15 minutes after the deposition crystallized the thin film.

In order to evaluate the electrical conductivity of the prepared thin film, the electrical resistance was measured in the measurement chamber 34. The measurement of the electrical resistance was performed after the sample was cooled to about room temperature following the annealing and transferred to the measurement chamber 34.

As in Embodiment 1, minimization of the electrical resistance was performed by Bayesian optimization. More specifically, the minimum value of common logarithm values of the resistance values was searched. Radial basis function (RBF) kernel was employed for kernel function, and 0.3 was employed for variance. Two numerical values 30 and 3 were used for a length scale. The narrower one of the credible intervals was employed. Lower confidence bound was employed for an acquisition function (A), and a minimum value is searched with A=−E+5σ. Assuming that the numerical range of oxygen partial pressure was 2.0×10⁻⁴ Pa to 5.0×10⁻³ Pa, the numerical range was divided into a 128 grid for search. The deposition conditions at the first three times were previously set to both ends of the grid (2.0×10⁻⁴ Pa and 5.0×10⁻³ Pa) and the center (2.51×10⁻³ Pa) so as to be sparse. The deposition conditions at the fourth time and after were decided according to Bayesian optimization and set so as not to select the same condition again.

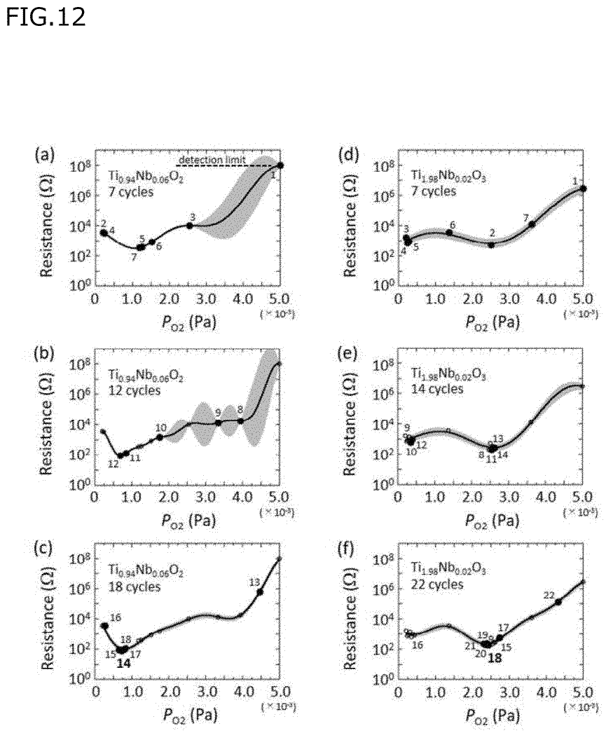

FIG. 12 is experimental data showing the oxygen partial pressure dependence of electrical resistance. Graphs (a)-(c) and (d)-(f) in FIG. 12 show experimental data obtained when Ti_{0.94}Nb_{0.06}O₂ and Ti_{1.98}Nb_{0.02}O₃ are employed as targets, respectively. The horizontal axis of each of the graphs represents oxygen partial pressure (Pa) and the vertical axis thereof represents electrical resistance (a). The respective points in the graphs represent search points according to Bayesian optimization, and a numerical value near each of the search points represents the number of searches. For example, the graphs (a)-(c) respectively show search points before 7th time, before 12th time and before 18-th time.

As shown in the graphs (a)-(c), if Ti_{0.94}Nb_{0.06}O₂ is employed for a target, the electrical resistance assumes the minimum value at the 14th time, and the oxygen partial pressure indicates substantially the same value at 15th time and after, so that the search is fully converged. Thus, the optimum oxygen partial pressure can be determined as 7.29×10⁻⁴ Pa. As shown in the graphs (d)-(f), if Ti_{1.98}Nb_{0.02}O₃ is employed for a target, the electrical resistance assumes the minimum value at the 18th time, and the search is converged when the oxygen partial pressure is 2.43×10⁴ Pa. The prediction curve obtained when Ti_{1.98}Nb_{0.02}O₃ is used is shifted more rightward than that obtained when Ti_{0.94}Nb_{0.06}O₂ is used, which is consistent with a less oxygen content in the target.

Two remarkable points can be found from the experiments. The first point is that the search for the correct minimum point was successful in the presence of two local minimum regions within the search region. The presence of two local minimum regions is derived from generation of a titanium reduction phase such as Ti₃O₅, Ti₂O₃ or the like. Nevertheless, the correct minimal value was successfully searched for.

The second point is that the time required for the experiments can be shortened to about a tenth in comparison with that required for experiments by hand. In this experiment, deposition was completed after about 24 hours per ten experiments, and optimization was completed in about one to two days. If the experiment is performed by a person, an average of two samples can be prepared per day, and 6 days are required for ten experiments. Since the duration of 6 days is evaluated assuming that the worker can be engaged in the experiment exclusively, 10 days or so may be required in view of time for other activities. This shows that the employment of this system can accelerate the research.

It should be considered that the embodiments disclosed this time are illustrative in all aspects and are not limitative. The scope of the present invention is indicated not by the meaning described above but by the claims, and all changes that fall within the meaning equivalent to the claims and the scope are to be embraced.

1-14. (canceled)

15. A sample preparation evaluation system comprising:
a preparation device that prepares a sample;
a measurement device that measures a physical property or a structure of the sample prepared by the preparation device and outputs substance information indicating the physical property or the structure of the sample; and
a prediction device connected to the preparation device and the measurement device, wherein
the prediction device predicts a preparation condition for optimizing the substance information based on a data set including the preparation condition of the sample and the substance information of the sample,
the preparation device prepares the sample according to the preparation condition predicted by the prediction device,
the prediction device adds the substance information of the sample and the preparation condition of the sample to the data set, and
the prediction device sequentially predicts the preparation condition based on the data set including the added preparation condition and the added substance information.

16. The sample preparation evaluation system according to claim 15, wherein
the prediction device predicts the preparation condition by performing Bayesian optimization based on the preparation condition and the substance information.

17. The sample preparation evaluation system according to claim 15, wherein
the prediction device predicts the preparation condition depending on a preparation capability related to the sample capable of being prepared by the preparation device.

18. The sample preparation evaluation system according to claim 15, further comprising a plurality of the measurement devices that respectively performs a plurality of types of measurement and outputs a plurality of types of substance information, wherein
the prediction device accepts an input of priorities of the plurality of types of the substance information, and
the prediction device predicts the preparation condition that optimizes the respective substance information according to the priorities.

19. The sample preparation evaluation system according to claim 15, wherein
the prediction device determines whether or not the data set satisfies a predetermined convergence condition, and converges sequential predictions of the preparation condition if determining that the convergence condition is satisfied.

20. The sample preparation evaluation system according claim 15, further comprising a management device that manages information on the sample prepared by the preparation device, wherein
the management device acquires the data set from the prediction device, stores the data set acquired in a storage unit, and outputs sample information including the preparation condition of the sample and the substance information of the sample from the storage unit in response to a request.

21. The sample preparation evaluation system according to claim 15, wherein
the prediction device accepts a setting input of an upper limit of a preparation amount of the sample, and
the prediction device determines that preparation of the sample is to be ended in the case where a preparation amount of the sample added to the data set reaches the upper limit.

22. The sample preparation evaluation system according to claim 15, wherein the preparation device marks the sample with identifying information capable of uniquely identifying the sample after completion of measurement by the measuring device.

23. The sample preparation evaluation system according to claim 19, wherein
the prediction device determines whether or not the prediction device predicts a same preparation condition a plurality of consecutive number of times,
the prediction device determines whether or not a measurement value of the substance information obtained from the measurement device is more proper than an prediction value of the substance information predicted when the prediction device predicts the preparation condition, and
the prediction device determines that the convergence condition is satisfied if determining that the preparation condition is predicted the plurality of consecutive number of times and that the measurement value is more proper than the prediction value.

24. The sample preparation evaluation system according to claim 20, wherein
the management device accepts a setting input of setting a type of a parameter included in the preparation condition and a type of the substance information, and
the management device instructs the prediction device to sequentially predict the preparation condition for optimizing the substance information of a set type, the preparation condition including a parameter of the set type.

25. The sample preparation evaluation system according to claim 20, wherein
the prediction device calculates a degree of correlation of each of a plurality of the parameters included in the preparation condition with the substance information based on the data set stored in the storage unit, and
the management device outputs information including a degree of correlation for each of the plurality of the parameters.

26. The sample preparation evaluation system according to claim 24, wherein
the management device accepts a setting input of setting an initial value of the data set with reference to the sample information stored in the storage unit, and outputs the data set to which the initial value is set to the prediction device, and
the prediction device starts prediction of the preparation condition based on the data set output from the management device.

27. A sample preparation evaluation method including a preparation device that prepares a sample, a measurement device that measures a physical property or a structure of the sample and outputs substance information indicating the physical property or the structure, and a prediction device connected to the preparation device and the measurement device, comprising:
predicting, by the prediction device, a preparation condition for optimizing the substance information based on a data set including the preparation condition of the sample and the substance information of the sample;
preparing, by the preparation device, the sample according to the preparation condition predicted by the prediction device;
measuring, by the measurement device, the substance information of the sample prepared:
adding, by the prediction device, the substance information measured by the measurement device and the preparation condition of the sample to be measured to the data set; and
sequentially predicting, by the prediction device, the preparation condition based on the data set including the added preparation condition and the added substance information.

28. A non-transitory computer-readable medium storing a computer program causing a computer to execute processing of:
requesting a management device that manages information on a sample measured by a measurement device that measures a physical property or a structure of the sample to output sample information including a preparation condition and substance information indicating the measured physical property or the measured structure of the sample,
displaying information output from the management device on a display unit,
accepting a setting input of setting a type of a parameter included in the preparation condition and a type of the substance information, and
requesting a prediction device that predicts the preparation condition for optimizing the substance information based on a data set including the preparation condition and the substance information to output the preparation condition and a type of the substance information that are selected and to sequentially predict the preparation condition in conjunction with the measurement device and a preparation device that prepares the sample measured by the measurement device.
